(19) United States
(12) Patent Application Publication
(10) Pub. No.: US 2006/0093663 A1
(43) Pub. Date: May 4, 2006

(54) Drug delivery system and drug capsule and transmitter used therefore

(75) Inventor: Masahiro Suzuki, Tokyo (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(73) Assignee: NEC Corporation

(21) Appl. No.: 11/264,022

(22) Filed: Nov. 2, 2005

(30) Foreign Application Priority Data
Nov. 4, 2004 (JP) 319989/2004
Sep. 7, 2005 (JP) 258638/2005

(51) Int. Cl. A61K 9/48; A61K 9/22

(52) U.S. Cl. 424/451; 604/890.1

(57) ABSTRACT

Release control of a drug in a drug capsule existing in a subject is extremely simply and accurately performed. A non-contact type IC chip which generates a voltage on the basis of a release command signal from the outside, and a piezoelectric actuator which is driven by the voltage, are attached on the surface of the drug capsule. The drug capsule is thrown into the subject by swallowing or the like. Then, the release command signal is radio-transmitted by a transmitter from the outside of the subject to the non-contact type IC chip at a predetermined timing, to perform drive control of the piezoelectric actuator, thereby making it possible to control separation or opening of the drug capsule, and to effect release control of the drug in the capsule.

### BACKGROUND OF THE INVENTION

1. Field of the Invention

The present invention relates to a drug delivery system, and to a drug capsule and a transmitter which are used for the drug delivery system. More particularly, the present invention relates to a drug delivery system configured to perform drug release control of a drug capsule in a subject, and to a drug capsule and a transmitter which are used for the drug delivery system.

2. Description of the Prior Art

It is effective for the purpose of treating and preventing various kinds of diseases or of promoting the growth and the like, to perform release control of a drug in a capsule after the drug capsule is swallowed by a person or poultry and is thrown into the body. For example, referring to National Publication of International Patent Application No. 2001-518492, there is disclosed a technique for performing control so that a drug filled in a capsule thrown or embedded in the body is released in stages. In this technique, a method is utilized in which a capsule cap is separated from a capsule body by using the osmotic pressure of the body fluid to the capsule.

Referring to Japanese Patent Laid-Open No. 6-23020, there is disclosed a technique which enables a drug in a capsule to be released in a limited part of the human body. That is, in this technique, an actuator for opening the capsule is used, and when the capsule reaches a desired part, a shape-memory alloy used as the actuator is heated so as to enable the capsule to be opened.

In the technique of International Patent Application No. 2001-518492 as described above, the osmotic pressure is used for opening the capsule. For this purpose, the body fluid needs to be used, and hence, the technique has an disadvantage that it is necessary to take the timing of foods and dosing into account, for example, and that in the case where the body fluid concentration is different from that assumed beforehand due to the individual difference, the body conditions and the like, it is difficult to release the drug in the timing as expected. Further, the technique of Japanese Patent Laid-Open No. 6-2320 as described above, has a disadvantage that since the shape-memory alloy is used, a member, a circuit and the time to heat the shape-memory alloy are needed, and the power consumption is also increased.

An object of the present invention is to provide a drug delivery system which enables drug release control of a drug capsule in a subject to be extremely simply performed by a command from the outside, and to provide the drug capsule and a transmitter which are used in the drug delivery system.

### BRIEF SUMMARY OF THE INVENTION

According to the present invention, there is provided a drug delivery system which performs release control of a drug in a subject, the drug delivery system comprising: a drug capsule having a non-contact type IC chip which generates a voltage on the basis of a release command signal from the outside, and an actuator which is driven by the voltage; and transmission means transmitting the release command signal to the non-contact type IC chip, wherein release control of the drug in the drug capsule is performed by drive control of the actuator.

According to the present invention, there is provided a drug capsule comprising a non-contact type IC chip which generates a voltage on the basis of a drug release command signal from the outside; and an actuator which is driven by the voltage, wherein drug release control is performed by drive control of the actuator.

According to the present invention, there is provided a transmitter used in a drug delivery system which includes a drug capsule having an actuator and a non-contact type IC chip generating a voltage for driving the actuator, and which performs release control of a drug by drive control of the actuator, the transmitter comprising means for generating a command signal for drug release control and for performing radio transmission of the command signal.

The operation of the present invention will be described below. The drug capsule having the non-contact IC chip which generates a voltage on the basis of the release command signal from the outside, and the actuator which is driven by the voltage is thrown into the subject by swallowing and the like. The release command signal is radio-transmitted in a predetermined timing by the transmitter from the outside of the subject to the non-contact type IC chip, so that the separation or the opening/closing of the drug capsule, or the jetting of the drug is controlled by drive control of the actuator, as a result of which the release control of the drug in the capsule is effected.

### BRIEF DESCRIPTION OF THE DRAWINGS

FIG. 1 is an overall view of an embodiment according to the present invention;

FIG. 2 is a plane view of a drug capsule in the embodiment according to the present invention;

FIG. 3 is a flow chart showing an example of the operation of the embodiment according to the present invention;

FIG. 4 is a figure showing an opened state of the drug capsule shown in FIG. 2, in which FIG. 4A is a part of a front view of the capsule, and FIG. 4B is a part of a longitudinal section of the capsule;

FIG. 5 is a flow chart showing another example of the operation of the embodiment according to the present invention;

FIG. 6 is a figure showing a drug case (capsule) of another embodiment according to the present invention, in which FIG. 6A is an overall perspective view of the drug case, and FIG. 6B is a part of a longitudinal section of the drug case;

FIG. 7 is a flow chart showing an example of the operation of another embodiment according to the present invention;

FIG. 8 is a flow chart showing another example of the operation of another embodiment according to the present invention; and

FIG. 9 shows a drug capsule of yet another embodiment according to the present invention, in which FIG. 9A is a side view in a state where a drive voltage is not applied to a piezoelectric actuator, and FIG. 9B is a side view in a state where the drive voltage is applied to the piezoelectric actuator.

### DETAILED DESCRIPTION OF THE PREFERRED EMBODIMENTS

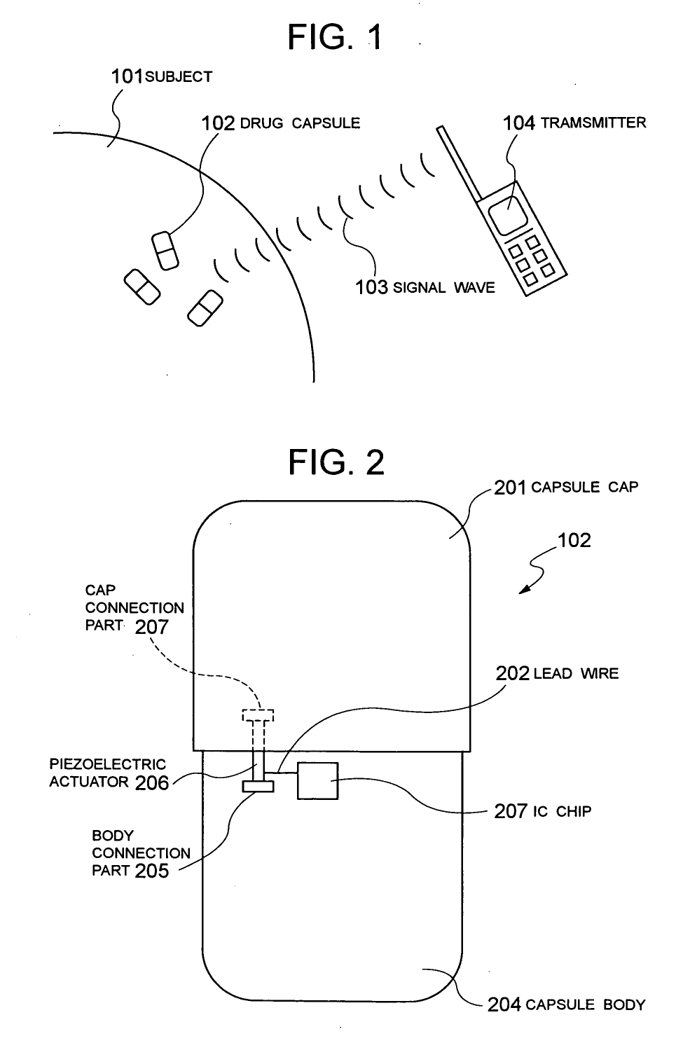

Embodiments according to the present invention are explained below with reference to the drawings. FIG. 1 is an overall view of an embodiment according to the present invention. In FIG. 1, a drug capsule 102 is assumed to exist in a subject 101 and a transceiver 104 is provided outside the subject 101. The separation or the opening of the drug capsule in the subject 101 is controlled by a signal wave (radio wave) 103 for drug release command from the transmitter 104, so as to effect the release control of the drug in the capsule.

An example of the drug capsule 102 is explained with reference to FIG. 2. In FIG. 2, the drug capsule 102 consists of a capsule cap 201 and a capsule body 204. The capsule cap 201 is made of an insoluble material, such as an insoluble mineral material, and is joined or bonded with the capsule body 204 so as to function as a cap of the drug capsule, and prevents a drug (not shown) filled in the capsule from leaking to the outside.

Similarly to the capsule cap 201, the capsule body 204 is made of an insoluble material, and is joined or bonded with the capsule cap 201, so as to have a function of holding the drug in the inside of the capsule.

An IC chip 203 is a non-contact type IC chip having a RF circuit, a control circuit, a capacitor for power supply, a memory or the like, and is attached to the outer surface of the capsule body 204. The IC chip 203 is connected to a piezoelectric actuator (piezoelectric element) 206 by a lead wire 202. When the IC chip 203 receives the signal wave 103 by radio from the outside (the outside of the subject 101) as shown in FIG. 1, current is generated inside the IC chip 203 so that a predetermined drive voltage is supplied to the piezoelectric actuator 206 through the lead wire 202.

The piezoelectric actuator 206 is a thin film type in which a piezoelectric element, such as for example a piezoelectric ceramic is formed in a bimorph structure, and is arranged to be physically deformed in the direction perpendicular to the film, when the predetermined voltage is applied to the piezoelectric actuator. As shown in FIG. 2, the piezoelectric actuator 206 is fixed to the outer surface of the capsule body 204 at the body connection part 205, and is fixed to the inner surface of the capsule cap 201 at the cap connection part 207. Thus, the piezoelectric actuator 206 is attached so that when the drive voltage is supplied from the IC chip 203 to the piezoelectric actuator 206 through the lead wire 202, the piezoelectric actuator 206 is distorted in the direction perpendicular to the surface of the capsule cap 201 and the capsule body 204.

Noted that in a state where no voltage is applied to the piezoelectric actuator 206, it has a planar form. In this planar form, as shown in FIG. 2, the cap connection part 207 which is an upper end part of the piezoelectric actuator 206 is bonded to the inner surface of the capsule cap 201, and the body connection part 205 which is the lower end part of the piezoelectric actuator 206 is bonded to the outer surface of the capsule body 204, so that the piezoelectric actuator 206 is sandwiched between the cap and the body. A sealing property is provided between the piezoelectric actuator 206, the capsule cap 201 and the capsule body 204 to such an extent that the leakage of the drug can be prevented.

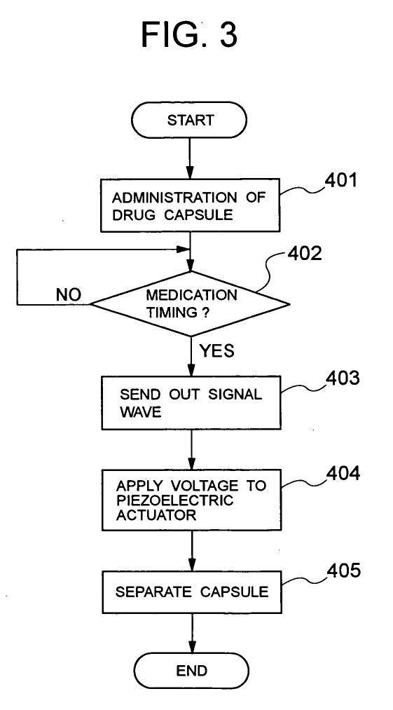

Next, an example of the operation of the present embodiment is explained using a flow chart shown in FIG. 3. The drug capsule 102 as shown in FIG. 2 is administered in the subject 101 by swallowing, embedding or the like (step 401). When the medication time is reached (step 402), the signal wave 103 is automatically or manually transmitted from the transceiver 104 to the drug capsule 102 in the body (step 403).

The signal wave 103 reaches the IC chip 203 of the drug capsule 102 in the body, so as to be received and converted to a current. The IC chip 203 generates a drive voltage for the piezoelectric actuator 206 on the basis of the current, and applies the drive voltage to the piezoelectric actuator 206 to drive it (step 404).

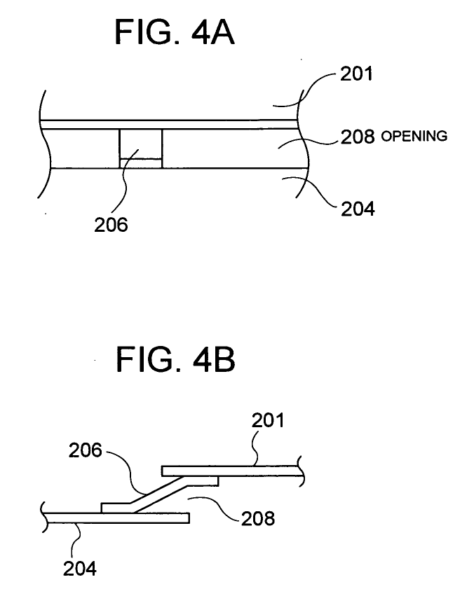

When the drive voltage is applied to the piezoelectric actuator 206, it is operated so as to be distorted in the direction perpendicular to the surface of the drug capsule 102 as described above, whereby an opening 208 which is a gap between the capsule body 204 and the capsule cap 201, is formed as shown in FIG. 4. Noted that FIG. 4A is a partially enlarged view of the opening 208 seen from the front, and is a figure showing a part of the piezoelectric actuator 206 seen from the lower side of FIG. 2. In addition, FIG. 4B shows a part of longitudinal section of the part of the piezoelectric actuator 206.

In this way, the opening 208 is formed between the capsule cap 201 and the capsule body 204, with the result that the drug is directly released from the opening, or that the body fluid flows into the capsule through the opening to increase the internal pressure in the capsule, and the capsule cap 201 is separated from the capsule body 204 under the action of the increase in the internal pressure (step 405), thereby enabling the drug to be released.

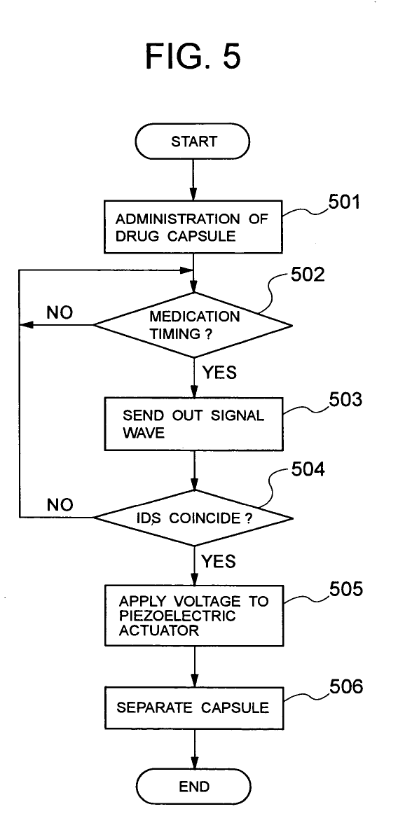

FIG. 5 is a flow chart showing an example of another operation of the present embodiment. In this example, a unique ID is assigned beforehand to the IC chip 203 attached to the drug capsule 102. The drug capsule 102 having such IC chip 203 is administered into the subject 101 by swallowing, embedding or the like (step 501). When the medication time is reached (step 502), the signal wave 103 including digital ID information is automatically or manually transmitted from the transceiver 104 to the drug capsule 102 in the body (step 503).

The signal wave 103 reaches the IC chip 203 of the drug capsule 102 in the body, so as to be received and converted to current and digital information. The IC chip 203 compares the ID which is the digital information with the ID assigned to the IC chip (step 504). When both of the IDs coincide with each other, the IC chip 203 applies a drive voltage to the piezoelectric actuator 206 to drive it (step 505). Thereby, the piezoelectric actuator 206 generates a distortion in the direction perpendicular to the surface of the drug capsule 102, so as to form the opening 208 between the capsule cap 201 and the capsule body 204, as shown in FIG. 4, with the result that the drug is directly released from the opening, or that the body fluid flows into the capsule through the opening to increase the internal pressure in the capsule, and the capsule is separated by the action of the increase in the internal pressure, thereby enabling the drug to be released (step 506).

In this example, the drug release control only for a specific drug capsule can be performed from the outside, so that it is possible to perform accurate drug administration.

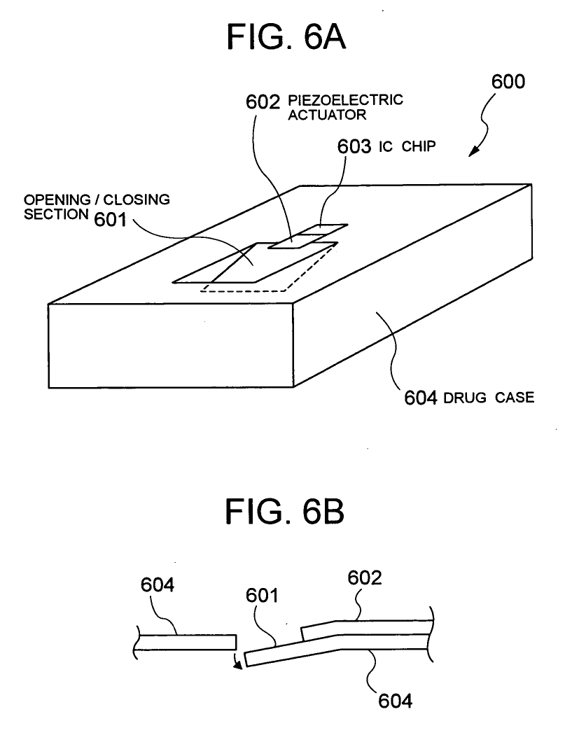

FIG. 6 is a figure showing another embodiment of the drug capsule according to the present invention. FIG. 6A is a perspective view of the drug capsule, and FIG. 6B is a longitudinal sectional view of the part of a piezoelectric actuator 602 in FIG. 6A. A drug capsule 600 in this example is provided with an opening/closing section 601, the piezoelectric actuator 602 and an IC chip 603 on the surface of a case 604. The opening/closing section 601 is supported at its one end side by the case 604, and is made of a material having flexibility.

Similarly to the example shown in FIG. 2, the piezoelectric actuator 602 is a thin film type formed in a bimorph structure or the like, and are bonded to the opening/closing section 601 and the case 604. The piezoelectric actuator 602 is also electrically connected to the IC chip 603. When no drive voltage is applied to the piezoelectric actuator 602, the opening/closing section 601 is in the closed state so that the drug in the case 604 is held. When the drive voltage is applied, the piezoelectric actuator 602 is distorted in the direction perpendicular to the case surface, and the opening/closing section 601 is made to be in the opened state as shown in FIG. 6B by this distortion force, thereby enabling the case 604 to be opened.

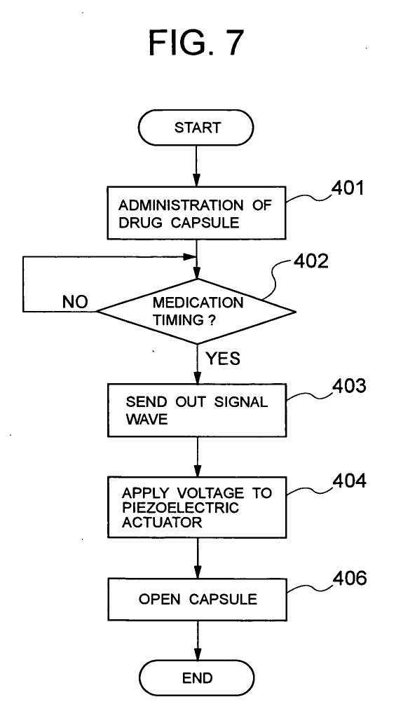

FIG. 7 is a flow chart showing an example of the operation of the embodiment shown in FIG. 6, according to the present invention, and corresponds to the operation of the flow chart of the previous embodiment shown in FIG. 3. In FIG. 7, steps equivalent to those in FIG. 3 are denoted by the same reference numerals. In this example, similarly to the operation from step 401 to step 404 shown in FIG. 3, when the voltage is applied to the piezoelectric actuator 602 (step 404), the opening/closing section 601 is made to be in the opened state, as shown in FIG. 6B, so that the drug in the drug case (capsule) 604 is released (step 406).

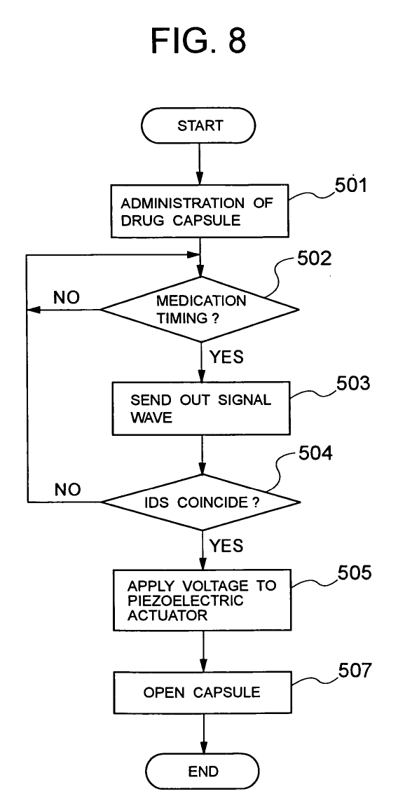

FIG. 8 is a flow chart showing an example of another operation of the embodiment shown in FIG. 6, according to the present invention, and corresponds to the operation of the flow chart of the previous embodiment shown in FIG. 5. In FIG. 8, steps equivalent to those in FIG. 5 are denoted by the same reference numerals. In this example, similarly to the operation from step 501 to step 505 shown in FIG. 5, when the voltage is applied to the piezoelectric actuator 602 (step 505), the opening/closing section 601 is made to be in the opened state, as shown in FIG. 6B, so that the drug in the drugs case (capsule) 604 is released (step 507).

In the embodiment shown in FIG. 6, the opening/closing section 601 which is provided for the drugs case 604, can be controlled to be opened/closed by performing drive control of the piezoelectric actuator 602, so that the opening/closing section 601 can also be made in the closed state by stopping the application of the drive voltage to the piezoelectric actuator 602. Accordingly, the opening/closing section 601 can be extremely finely controlled by the external signal wave (radio wave) 103, which results in a new effect that drug release control can be easily performed in stages (in step by step manner).

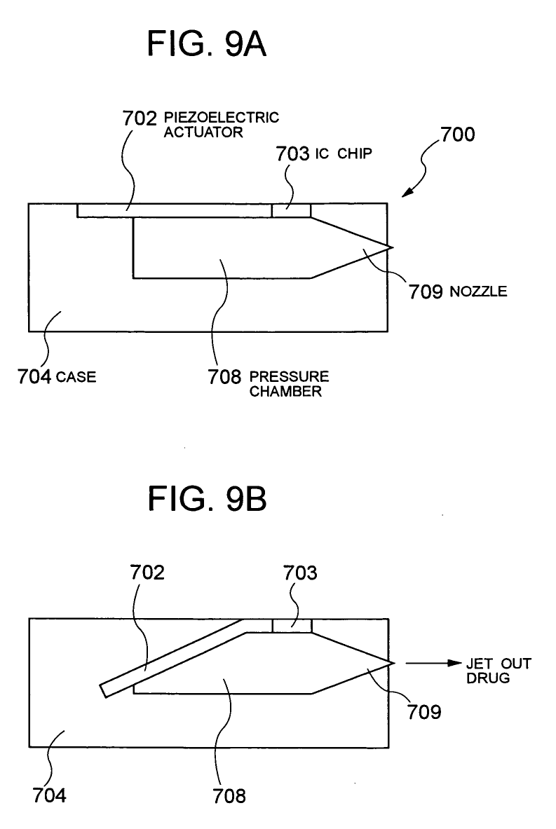

FIG. 9 is a side view showing yet another embodiment of the drug capsule according to the present invention. FIG. 9A is a side view in a state where the drive voltage is not applied, and FIG. 9B is a side view in a state where the drive voltage is applied. In a drug capsule 700 of this example, a piezoelectric actuator 702 is at its one end, electrically connected to an IC chip 703, and supported by a case 704, so as to constitute a cantilever structure.

In the case 704, a pressure chamber 708 filled with a liquid drug is provided. The pressure chamber 708 is made of a material having enough flexibility to be contracted by a pressure caused by the deformation of the piezoelectric actuator 702. In front of the pressure chamber 708, there is provided a nozzle section 709 at the tip of which a minimal hole is bored. The hole is sized so that when the pressure is not applied to the pressure chamber 708, the drug is prevented from flowing to the outside through the hole due to the surface tension of the drug, and that when the pressure is applied to the pressure chamber 708, the drug is made to jet out.

When the drive voltage is not applied to the piezoelectric actuator 702, the piezoelectric actuator 702 as a cantilever remains flat with respect to the pressure chamber 708, as shown in FIG. 9A, and hence, any pressure is not applied to the pressure chamber 708. Thus, the drugs filled in the pressure chamber 708 is not made to jet out from the hole of the nozzle 709.

When the drive voltage is applied to the piezoelectric actuator 702, as shown in FIG. 9B, the piezoelectric actuator 702 as the cantilever generates the distortion in the downward direction in the figure. Under the effect of the force caused by this distortion, the pressure chamber 708 is made to contract and thereby the pressure in the pressure chamber is increased. As a result, the drug is made to jet out from the hole at the tip of the nozzle 709.

Noted that the drug release control operation for the drug capsule 700 is similar to the previous embodiment, and hence, it is possible to arrange that the IC chip 703 is provided with a specific ID, and when the ID information included in the signal wave 103 coincides with the ID specific to the IC chip 703, the drive voltage for the piezoelectric actuator 702 can be generated. Further, it is obvious that the amount of deformation of the piezoelectric actuator 702 can be finely controlled by the external signal wave (radio wave) 103, and the drug release control in step by step manner is also possible.

According to the present invention, a non-contact type IC chip and an actuator are provided for a capsule, and a command signal is transmitted by radio from the outside, i.e. from the outside of a subject, to drive the actuator, thereby making it possible to control the opening and separation of the capsule, the jetting of a drug, and the like. As a result, an effect that release control of the drug can be extremely simply performed, is obtained.

What is claimed is:

1. A drug delivery system which performs release control of a drug in a subject, the drug delivery system comprising: a drug capsule having a non-contact type IC chip which generates a voltage on the basis of a release command signal from the outside, and an actuator which is driven by said voltage; and transmission means transmitting said release command signal to said non-contact type IC chip, wherein release control of the drug in said drug capsule is performed by drive control of said actuator.

2. The drug delivery system according to claim 1, wherein a specific ID is assigned to said non-contact type IC chip, wherein said transmission means is constituted to transmit ID information together with said release command signal, and wherein said non-contact type IC chip receives said ID information and generates said voltage, when the ID information coincides with the specific ID assigned to the non-contact type IC chip.

3. The drug delivery system according to claim 1, wherein said transmission means is constituted to generate a command signal for effecting and stopping the generation of said voltage.

4. The drug delivery system according to claim 3, wherein the release control of said drug is performed in step by step manner by the command signal for effecting and stopping the generation of said voltage.

5. A drug capsule comprising: a non-contact type IC chip which generates a voltage on the basis of a drug release command signal from the outside; and an actuator which is driven by said voltage, wherein drug release control is performed by drive control of said actuator.

6. The drug capsule according to claim 5, wherein said IC chip having a specific ID assigned thereto receives ID information transmitted together with said release command signal and generates said voltage when the ID information coincides with the specific ID assigned to the IC chip.

7. The drug capsule according to claim 5, further comprising a capsule body and a capsule cap, wherein a gap between said capsule body and said capsule cap is formed by driving said actuator.

8. The drug capsule according to claim 5, further comprising a capsule case having an opening/closing section, wherein open/close control of said opening/closing section is performed by drive control of said actuator.

9. The drug capsule according to claim 5, further comprising a flexible pressure chamber in which a drug is filled and which has a nozzle capable of jetting out the drug, wherein a pressure is applied to said pressure chamber by drive control of said actuator to effect release control of said drug.

10. A transmitter used in a drug delivery system which includes a drug capsule having an actuator and a non-contact type IC chip generating a voltage for driving the actuator, and which performs release control of a drug by drive control of said actuator, said transmitter comprising means for generating a command signal for drug release control and for performing radio transmission of the command signal.

11. The transmitter according to claim 10, wherein the radio transmitter also transmits ID information specifying a non-contact type IC chip.
